(19) United States
(12) Patent Application Publication
(10) Pub. No.: US 2002/0153010 A1
(43) Pub. Date: Oct. 24, 2002

(54) System and method for total liquid ventilation with very low priming volume

(76) Inventors: Allan L. Rozenberg, San Diego, CA (US); John K. Hoffman, Poway, CA (US)

Correspondence Address: KNOBBE MARTENS OLSON & BEAR LLP, 2040 MAIN STREET, FOURTEENTH FLOOR, IRVINE, CA, 91614, US

(21) Appl. No.: 09/840,544

(22) Filed: Apr. 23, 2001

(51) Int. Cl. A61M 11/00; A61M 15/00; A61M 16/10

(52) U.S. Cl. 128/203.12; 128/913

(57) ABSTRACT

A liquid ventilation apparatus and method for facilitating liquid breathing by a patient. The apparatus can include a pressurizable chamber adapted to contain a breathable liquid and a gas, such that pressurizing and depressurizing the chamber moves a breathable liquid into and out of the lungs of a patient.

### BACKGROUND OF THE INVENTION

1. Field of the Invention

The present invention relates to apparatus for performing total liquid ventilation on patients. More particularly, the invention is related to inspiration and expiration systems for liquid ventilation.

2. Description of the Related Art

Liquid ventilation is a procedure involving temporarily filling pulmonary air passages with an oxygenated liquid medium. It was first demonstrated that mammals submerged in hyperoxygenated saline could breathe liquid and successfully resume gas breathing in 1962. However, this approach to liquid ventilation (LV) was eventually abandoned, due to the practical difficulties of dissolving sufficient quantities of O₂ in saline (even at hyperbaric pressures), and because saline rinses away much of the surfactant lining the lung alveoli. These problems were overcome in 1966 by Dr. Leland Clark, who was the first to use perfluorocarbon liquids (now oxygenated at atmospheric pressure) to support the respiration of mice, cats, and dogs.

Perfluorocarbon (PFC) liquids are derived from common organic compounds by the replacement of all carbon-bound hydrogen atoms with fluorine atoms. These liquids are clear, colorless, odorless, nonflammable, and essentially insoluble in water. PFC liquids are denser than water and soft tissue, and have low surface tension and, for the most part, low viscosity. Perfluorocarbon liquids are unique in their high affinity for gases, dissolving more than 20 times as much O₂ and over 3 times as much CO₂ as water. Like other highly inert fluorocarbon materials, perfluorocarbon liquids are extremely nontoxic and biocompatible.

In particular, perfluorocarbon liquid ventilation is a promising treatment of respiratory distress syndromes involving surfactant deficiency or dysfunction. Elevated alveolar surface tension plays a central role in the pathophysiology of the Respiratory Distress Syndrome (RDS) of prematurity and is thought to contribute to lung dysfunction in the Adult Respiratory Distress Syndrome. Perfluorocarbon liquid ventilation is effective in surfactant-deficient premature animals because it eliminates air/fluid interfaces in the lung and thereby greatly reduces pulmonary surface tension. Liquid ventilation can be accomplished at acceptable alveolar pressures without impairing cardiac output and provides excellent gas exchange even in very premature animals.

Currently, in total liquid ventilation, perfluorocarbon liquid is extracorporeally oxygenated and purged of carbon dioxide, and tidal breaths of the liquid are mechanically cycled into and out of the lungs. Many investigators have utilized gravity-induced ventilation whereby reservoirs above and below the level of the patient's lungs induce inspiration and expiration respectively.

As an alternative to such gravity based systems, pumped liquid ventilators have been used. However, these systems have generally been complex in nature, having one or more mechanical pumps and fluorocarbon reservoirs in order to provide sufficient user control over inspiration, expiration, tube priming, and other functions performed by the ventilation system. These systems also generally have separate reservoirs and gas exchangers, which requires an increased PFC priming volume and increases the loss of PFC.

Unfortunately, perfluorocarbon liquids are relatively expensive and current systems require large priming volumes due to their large and complex designs. Further, large amounts of the expensive PFCs are lost with current systems. In many systems, the greatest losses occur as the circulated liquid medium is subject to extracorporeal oxygenation and carbon dioxide purging. In particular, a great deal of gaseous oxygen must be introduced into the respiratory promoter (breathable liquid) to disassociate and purge the accumulated carbon dioxide prior to reintroduction of the respiratory promoter into the body. The majority of the oxygen passes through the respiratory promoter and is vented, carrying with it carbon dioxide and, unfortunately, fluorochemical vapor. Of course, if the therapy is to be continued additional respiratory promoter must be added to maintain effective residual volumes. As fluorochemical liquids and other respiratory promoters suitable for liquid ventilation can be relatively expensive, such losses can substantially raise the cost of such therapies. Moreover, the loss of respiratory promoter complicates both dosing regimens and monitoring the current volume of material in the lung.

Besides the loss of expensive fluorochemical, the use of fluorochemical based respiratory promoters can damage conventional ventilation equipment which incorporate materials that are not compatible. For example, a number of engineering plastics used in current ventilators tend to swell in the presence of fluorochemicals. In other currently used materials, exposure to fluorochemicals will leach plasticizers causing the material to become brittle and subject to failure under much less stress. Further, modem conventional ventilators contain a number of delicate sensors for monitoring the levels and condition of both the inspiratory and respiratory gases. As with the ventilators themselves, many of these sensors incorporate materials that are not fully compatible with fluorochemicals or other potential respiratory promoters. Accordingly, the use of fluorochemicals with conventional systems may lead to a degradation of sensory data and inaccurate readings if the apparatus is not properly monitored and maintained. Such materials problems can be severe handicaps when trying to gain regulatory approval of a therapeutic method or incorporation of a specific device into a preapproved treatment.

### SUMMARY OF THE INVENTION

In one embodiment the present invention relates to a liquid ventilation apparatus for facilitating liquid breathing by a patient. The apparatus can include a chamber adapted to contain a breathable liquid and a gas; a gas supply system connected to the chamber and adapted to supply an oxygen-carrying breathing gas into the chamber under pressure, thereby pressurizing the breathable liquid; a gas removal system connected to the chamber and adapted to remove gas from the chamber; a gas flow control system controlling the gas supply system and the gas removal system to alternately pressurize and depressurize the chamber; a liquid supply line exiting the chamber and adapted to carry breathable liquid from the chamber to a patient when the chamber is pressurized; and a liquid return line communicating with the chamber and adapted to carry breathable liquid from a patient to the chamber when the chamber is depressurized.

The apparatus can include at least a first valve associated with at least one of the liquid supply line and the liquid return line so that liquid flows only outward from the chamber through the liquid supply line or only inward toward the chamber through the liquid return line. The first valve can be a valve connected to both the liquid supply line and the liquid return line, and can be adapted to permit fluid communication between one, but not both, of the lines and the lungs of a patient at any one time. The first valve can be a “Y” valve, for example. The apparatus can include a second valve, wherein the first valve is a check valve controlling one-way flow through the liquid supply line and the second valve is a check valve controlling one-way flow through the liquid return line. The apparatus can include a second valve, wherein the first valve is a pinch valve controlling one-way flow through the liquid supply line and the second valve is a pinch valve controlling one-way flow through the liquid return line.

The pressurization of the chamber can drive liquid flow out from the chamber through the liquid supply line and depressurization of the chamber can draw liquid into the chamber through the liquid return line. For example, the depressurization of the chamber can reduce the pressure in the chamber to a level no greater than atmospheric pressure. The depressurization of the chamber can reduce the pressure in the chamber to a pressure less than atmospheric pressure.

The gas flow control system can include a flow controller adapted to control the flow of a gas into or out of the chamber. The flow controller can be, for example, a mass flow controller. The flow controller can be, for example, a digital flow controller.

The apparatus further can include a safety system. The safety system can include, for example, a safety valve. The safety system can include a rupture device, for example.

The apparatus further can include a liquid conservation device. The liquid conservation device is can be, for example, condenser. The conservation device can be associated with the gas removal system, for example.

The apparatus further can include a decontamination device for removing contaminants from the breathable liquid or gas. The decontamination device can be, for example, a filter. The can be an aqueous trap. The decontamination device can be associated with the liquid supply line to decontaminate the breathable liquid. The decontamination device can be associated with the liquid return line to decontaminate the breathable liquid.

The apparatus further can include a heating device to warm the breathable liquid. The heating device can be associated with the chamber. The heating device can warm the breathable liquid to body temperature, for example.

The chamber further can include a spray nozzle by which the breathable liquid is reintroduced into the chamber such that gas exchange occurs. The chamber can include a gas diffuser connected to the gas supply system such that the oxygen-carrying breathing gas is diffused into the breathable liquid. The gas diffuser can be an airstone, for example. The chamber can include a hold-up device to facilitate gas exchange, for example. The chamber can be sealed. The chamber can be a column gas exchanger.

The apparatus can include a volume of the breathable liquid. The volume of the breathable liquid can be approximately equal to the functional residual capacity of the patient. The volume of the breathable liquid can be approximately equal to at least the functional residual capacity of the patient, a priming volume, and a tidal volume.

The breathable liquid can be, for example, a perfluorochemical (PFC). Examples of the PFC can include C-75, FC-77, RM-101, Hostinert 130, APF-145, APF-140, APF-125, perfluorodecalin, perfluorooctyl bromide, perfluorobutyltetrahydrofuran, perfluoropropyl-tetrahydropyran, dimethyladamantane, trimethyl-bicyclo-nonane, and mixtures thereof.

The oxygen containing breathing gas can include, for example, oxygen as a first gas and a second gas which can include nitrogen, helium, argon, nitric oxide, and the like. The gas can be a bioactive agent, respiratory promoter, and the like.

The gas removal system can include a vacuum source. For example, the vacuum source may be a wall source vacuum. The vacuum source may be a vacuum pump, for example.

The liquid supply line can include a flexible tubing. The flexible tubing can be urethane, viton, and the like. The liquid return line can include a flexible tubing. The flexible tubing can be urethane, viton, and the like.

The apparatus further can include an endotracheal tube for delivering the breathable liquid to the patient and for conveying the breathable liquid from the patient.

The apparatus further can include an automated control system configured to control the activity of a valve, a flow control system, and the like. The control system can include a computer, other programmable controller, and the like.

The chamber can be a reservoir and a gas exchanger.

The apparatus further can include a liquid introduction port. The liquid introduction port can be a stopcock, a three-way valve, and the like.

The various specific features discussed in relation to the apparatus, above, can also be used in the additional embodiments, including the methods described below.

Another embodiment of the present invention relates to a method for providing total liquid ventilation to a patient. The method can include providing a liquid ventilation apparatus having a chamber containing a volume of a breathable liquid; delivering the breathable liquid from the liquid ventilation apparatus to a patient by pressurizing the chamber; and then moving the breathable liquid from the patient to the liquid ventilation apparatus by depressurizing the chamber.

Further, the delivery step can be accomplished through a first conduit and the moving step can be accomplished through a second conduit. The flow of the breathable liquid can be selectively directed through the first or the second conduit by means of a valve. The valve can be a check valve, an externally actuated valve, and the like.

A further embodiment of the present invention relates to a method for performing full tidal liquid ventilation on a patient without the use of liquid pumps. The method can include pressurizing a breathable liquid-containing chamber by introducing oxygen containing breathing gas thereinto, thereby to move the breathable liquid into a patient; and depressurizing the chamber by removing gas therefrom, thereby to move breathable liquid from the patient into the chamber.

Additionally, the breathable liquid can be moved from the chamber into the patient at least in part through a first conduit and the breathable liquid can be moved from the patient to the chamber at least in part through a second conduit. The breathable liquid can be selectively directed through the first or the second conduit by a valve.

### BRIEF DESCRIPTION OF THE DRAWINGS

FIG. 1 is a schematic illustration of a liquid breathing apparatus according to the present invention.

FIG. 2 is a schematic illustration of a preferred embodiment of a total liquid ventilator according to the present invention.

FIGS. 3 is a schematic illustration of a preferred embodiment of the present invention with a control system.

### DETAILED DESCRIPTION OF THE PREFERRED EMBODIMENT

Preferred embodiments of the present invention will now be described with reference to the accompanying Figures, wherein like numerals refer to like elements throughout. The terminology used in the description presented herein is intended to be interpreted in its broadest reasonable manner, even though it is being utilized in conjunction with a detailed description of certain specific preferred embodiments of the present invention. This is further emphasized below with respect to some particular terms. Any terminology intended to be interpreted by the reader in any manner more restrictive than that provided by its ordinary meaning will be overtly and specifically defined as such in this specification.

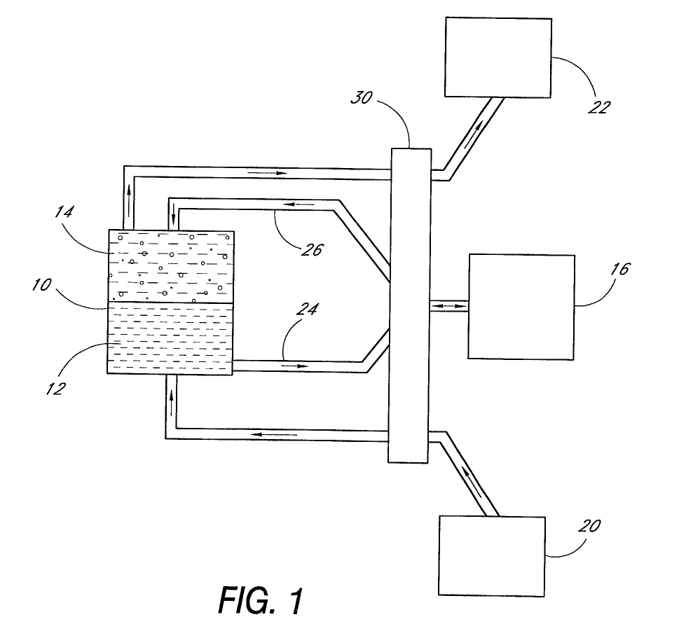

Referring now to FIG. 1, one embodiment of a liquid breathing apparatus incorporating the principles of the present invention is illustrated. The system of FIG. 1 comprises a chamber 10 adapted to hold a breathable liquid 12 and a head-space gas 14. In preferred embodiments the chamber 10 is sealed. The chamber 10 can be any size or shape and can be made of any suitable material. In preferred embodiments the chamber 10 can be tall and narrow, e.g., the height can be 2, 3, 4, 5, or more times greater than the diameter or width. One skilled in the art can easily select a material, but in particular may consider the compatibility of the material with the gases and breathable liquids 12. In selecting a chamber of an appropriate size and shape, one skilled in the art may consider, for example, volume requirements, pressure demands, gas exchange efficiency, and working space limits (practical size of hospital rooms, etc.). As will be discussed more fully below, the volume of breathable liquid 12 required for a patient 16 will depend upon the needs and situation of the particular patient. The pressure that the chamber 10 must be able to support will also influence the size and shape of the chamber 10. Preferably, the chamber can withstand an internal pressure of at least 1, 2, 3, 4, or more atmospheres.

As used herein, the breathable liquid 12 can include any gas dissolving liquid or like material. In preferred embodiments the breathable liquid 12 comprises a fluorocarbon. Specific embodiments of suitable gas carrying liquids are well known to those of skill in the art, with the bis(F-alkyl) ethanes such as F-44E and F-66E, as well as perfluorodecalin and perfluorooctyl bromide (Perflubron, Liquivent™, Alliance) being examples of suitable fluorocarbons for pulmonary oxygen delivery and carbon dioxide removal. Other examples of suitable perfluorocarbons include FC-77 (3M), FC-75 (3M), RM-101 (Miteni), APF-140 (Air Products), FC-75, FC-77, RM-101, Hostinert 130, APF-145, APF-140, APF-125, perfluorobutyltetrahydrofuran, perfluoropropyl-tetrahydropyran, dimethyladamantane, trimethyl-bicyclo-nonane, and mixtures thereof. Accordingly, for the purposes of the instant application the terms “breathable liquid” and “fluorocarbon” shall be used interchangeably unless indicated otherwise by the contextual placement, with fluorocarbons representing a preferred breathable liquid.

It should be noted that fluorocarbons can cause chemicals and plasticizers to leach out of certain plastics and other materials. Thus, any parts that come into contact with fluorocarbons should be compatible with the liquid and safe for patient contact.

As stated above, the volume of the breathable liquid 12 depends upon the particular patient 16. The required volume for the system also can depend upon the volume required to prime the system. Where the distance from the chamber 10 to the patient 16 is short, then less volume is required. Conversely, where the distance is greater, then a larger volume of breathable liquid 12 is required. Thus, in preferred embodiments the minimum required volume can be the functional residual capacity of the patient 16 plus the priming volume of the system, plus a safety factor of 10%, 20%, 30%, 40%, 50% or more of the functional residual capacity. Specifically, in some embodiments the volume is from 50 to 3000 mL. In other preferred embodiments the volume of breathable liquid can be approximately equal to at least the functional residual capacity of the patient, a priming volume, and a tidal volume. In another embodiment the volume of breathable liquid can be approximately equal to the functional residual capacity of the patient.

The head-space gas 14 includes an oxygen-carrying breathing gas from the gas supply system 20, as well as any other desired biocompatible gas, including, e.g., nitrogen and water vapor. In one embodiment the oxygen carrying breathing gas from the gas supply system 20 enters the chamber 10 and oxygenates the breathable liquid 12 while facilitating carbon dioxide removal therefrom. A quantity of the oxygen-carrying breathing gas passes through the breathable liquid 12 and associates with and purges carbon dioxide as well as any other gases expired from the patient 16. This combination of gases and vapors is referred to collectively as the head-space gas 14.

The gas supply system 20 connected to the chamber 10 supplies the oxygen-carrying breathing gas to the breathable liquid 12 and can act both to oxygenate and also to purge other expired gases, such as carbon dioxide from the breathable liquid 12. The oxygen-carrying breathing gas can be pure oxygen or a mixture of oxygen and other gases or therapeutic agents that may be desired for the lungs of a patient 16. One skilled in the art will understand that the gas supply system 20 can use numerous sources and combinations of devices to provide the desired gas to the chamber 10. In a preferred embodiment the gas supply system 20 can include internally supplied gas from a wall source, for example. Many hospitals, medical offices and care centers provide such gas supply systems 20. In other embodiments the gas supply system 20 utilizes tanks or canisters containing the desired gas. Further, a gas compressor can be used to supply gas.

The head-space gas 14 exits the chamber 10 by the gas removal system 22. In preferred embodiments, the gas removal system 22 comprises a vacuum source. Examples of possible vacuum sources include vacuum pumps and wall sources, such as found in many hospitals and medical offices.

The gas flow control system 30 controls the gas supply system 20 and the gas removal system 22 to provide and remove gas and to alternately pressurize and depressurize the chamber 10. The gas flow control system 30 can include different kinds of flow controllers, including, for example, mass flow controllers, digital flow controllers, and the like. A large variety of mass flow controllers are commercially available and within the understanding of those skilled in the art. Digital flow controllers can include a valve with a fixed orifice or a number of valves with different fixed orifices in parallel to each other, which determine the volume of gas that flows into or out of the chamber 10. One of skill in the art can easily select an appropriate flow controller or combination of flow controllers for use in the present invention.

In embodiments of the invention a single mass flow controller on the gas supply system 20 can be used in combination with a digital flow controller on the gas removal system 22, for example. In preferred embodiments two or more mass flow controllers can be utilized, with at least one on the gas supply system 20 and at least one on the gas removal system 22. In other embodiments the vacuum pull and/or the supply flow can be adjusted with respect to the other. Other configurations are well known to those of skill in the art.

As noted above, the gas flow control system 30 controls the gas supply to and removal from the chamber 10. The gas flow control system also controls the pressurization and depressurization of the chamber 10. When the chamber 10 is pressurized, a volume of the breathable liquid 12 is carried through the liquid supply line 24 to the patient 16. When the chamber 10 is depressurized, the volume of the breathable liquid 12 is carried from the patient 16 back through the liquid return line 26 to the chamber 10.

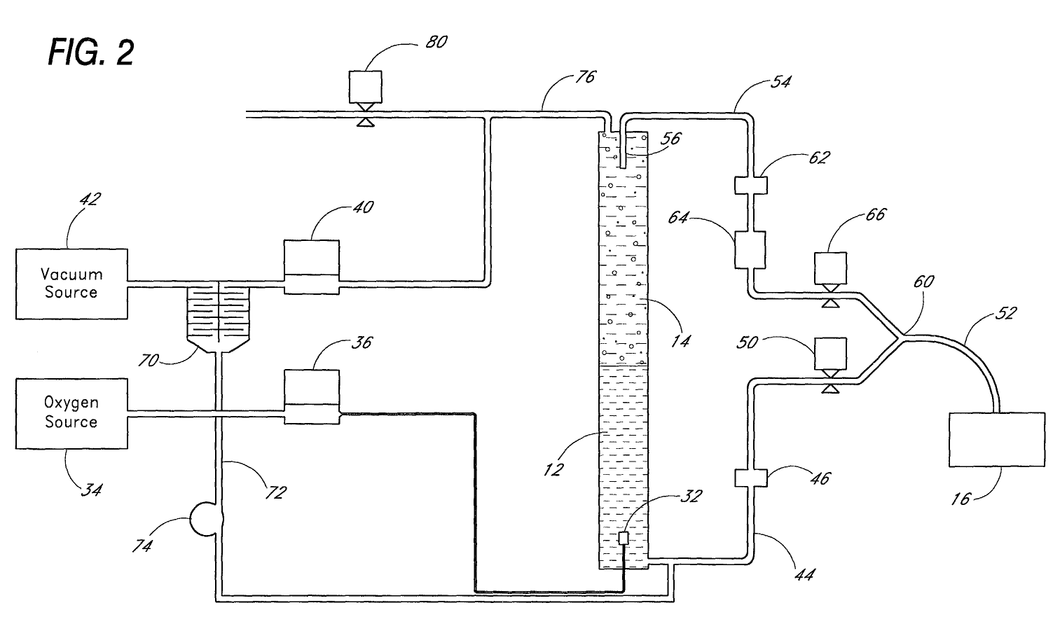

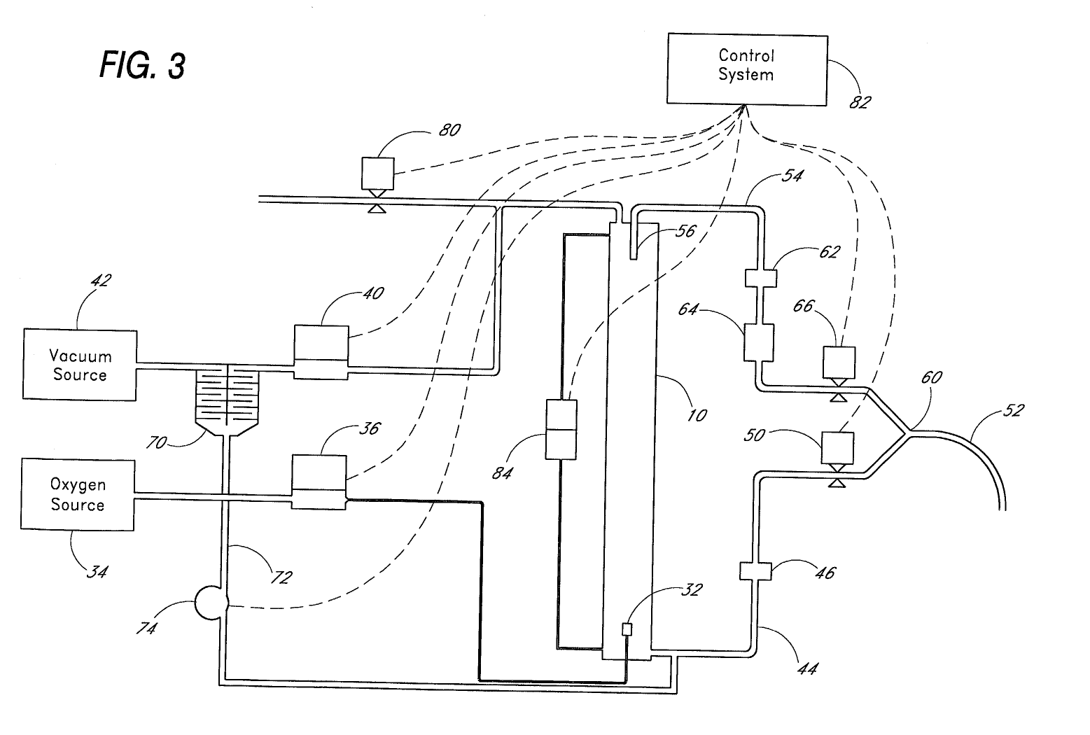

Having thus described the general principals of a preferred embodiment of the present invention, a more detailed illustration of such a system is present in FIGS. 2-3. Referring now to one embodiment of the invention depicted in FIG. 2, the chamber 10 can be a sealed column gas exchanger. An oxygen source 34 supplies oxygen to the chamber 10. Although FIG. 2 depicts an oxygen source 34, it should be understood that other gases and mixtures of gases and vapors can be used as well. The oxygen enters the chamber 10 through an airstone 32, which diffuses the oxygen as it enters the chamber 10 and mixes with the breathable liquid 12. The airstone 32 is one example of a device that can be used to diffuse the gas in order to facilitate gas exchange and carbon dioxide purging in the breathable liquid 12.

The head-space gas 14 can be removed by a vacuum source 42. En route to the vacuum source 42, the gas 14 removed from the head-space can pass through a condenser 70. Within the condenser 70, liquid vapor, such as vapor from the breathable liquid 12, condenses and is then pumped by pump 74 back to the chamber 10 via a conduit 72. Condensed water vapor is preferably removed by any conventional means, such as by flotation filtration or desiccation. The pump can be any commercial or custom made variety including a peristaltic pump, a diaphragm pump, a piston pump and the like.

A flow controller 36 provides a controllable flow of oxygen-carrying breathing gas to the chamber 10 from the oxygen source 34. Another flow controller 40 connected to the vacuum source 42 provides a controlled flow of head-space gas 14 out of the chamber 10. Many types of flow controllers can be used and they can be used in various numbers and combinations. For example, either or both flow controllers 36, 40 can be one or more mass flow controllers, digital flow controllers, or the like. Further, in one embodiment a digital flow controller can be used rather than a mass flow controller if the flow controller is likely to come into contact with a liquid, because the liquid may damage the mass flow sensor of the mass flow controller.

The breathable liquid 12 is supplied to the patient 16 through a conduit 44 that is connected from the bottom of the chamber 10 to the patient 16 through a filter 46 and a first valve 50 (such as a pinch valve, a check valve, or a mechanical valve) to a connector 60 at an endotracheal tube 52 hub. The expired breathable liquid 12 is returned to the chamber 10 from the patient 16 through endotracheal tube 52 to the connector 60 through a conduit 54, a second valve 66, an aqueous trap 64, a filter 62, and into the chamber 10, preferably through a spray nozzle 56.

The conduits 44, 54 are selected based upon compatibility with the breathing liquid 12 and any gases or therapeutics used in the apparatus. They can include flexible tubing (i.e. polymeric or plastic tubing) which are well known to those of skill in the art, and which are also suitable for gravity feed and multi-pump liquid ventilators. However, the term conduit is intended to include any type of pipe, channel, or other flexible or rigid device creating a passage through which liquid may flow. Fluorochemicals can leach chemicals and plasticizers from various types of tubing or hoses. Thus, a conduit of fluorocarbon-compatible material should be selected, such as urethane, viton and the like. The size or diameter and the length of the tubing can be selected based upon the volume of liquid that is being delivered to the patient and the location of the patient relative to the liquid ventilator of the present invention.

The conduits 44, 54 each connect to communicate with the lungs of the patient 16, preferably through an endotracheal tube 52. The connection may be facilitated by a connector 60. The connector 60 typically comprises a material and configuration well known to those of skill in the art, for example, a T or Y connector.

The endotracheal tube 52 has one end inserted into the trachea of a patient 16, for delivering the inspired breathable liquid 12 and removing expired breathable liquid 12 from the patient's pulmonary air passages. The endotracheal tube 52 typically comprises a conduit made of polymer tubing of material and configuration well known to those of skill in the art.

The filters 46, 62, likewise can be selected based upon compatibility with the materials used in the invention. Filters can be obtained from numerous commercial sources. One skilled in the art can easily select suitable filters based upon the particular materials being used in a system. In a preferred embodiment a filter equivalent to a conventional blood filter can be used.

The aqueous trap 64 captures aqueous material expired from the patient 16. In one preferred embodiment a custom made trap 64 captures aqueous material as the breathable liquid 12 passes through the trap during expiration from the patient 16. Any aqueous waste is trapped (e.g., by flotation, since fluorocarbons are more dense than water) and the breathable liquid 12 returns to the chamber 10.

In one embodiment, inspiration occurs when the valve 50 is opened while the valve 66 remains closed and the flow of oxygen is set higher than the gas flow to the vacuum source 42. This pressurizes the chamber 10 forcing the breathable liquid 12 into the lungs of the patient 16. Any type of valve can be used. For example, the valves 50, 66 can be pinch valves and can be custom made, pneumatic or electric. One skilled in the art will recognize that various valve configurations and combinations can be utilized in the present invention. One can easily select a valve or combination of valves in order to carry out the purposes of the system. For example, one or more check valves may be used. In another embodiment a mechanical “Y” valve can be used; i.e., a valve that can mechanically connect one conduit to a selected one of two other conduits (but not both other conduits), and is typically arranged, e.g., in a “Y” configuration with a rotatable element in the valve body having a curved or angled channel therethrough to provide fluid communication between a selected two of the legs of the “Y”.

During expiration the valve 50 remains closed while the valve 66 is opened. The flow of oxygen is set lower than the flow to the vacuum source 42 so that the chamber 10 is depressurized such that the breathable liquid 12 is expired from the lungs of the patient 16. As noted previously, the gas flow control system 30 (see FIG. 1) controls the gas supply system 20 and gas removal system 22 to pressurize and depressurize the system. While not inspiring or expiring, the oxygen flow and vacuum flow can be set to be equal, which allows the breathable liquid 12 to be oxygenated without causing liquid to flow into or out of the lungs of the patient 16.

In embodiments of the present invention the chamber 10 and other components of the apparatus can withstand a range of pressures. During depressurization the pressure in the chamber can be more than, equal to, or less than atmospheric pressure. For example, the pressure in the chamber 10 during pressurization can advantageously be 0, 10, 50, 100, 200, 400, 600, 760, or 1000 torr.

As stated above, the breathable liquid 12 can be reintroduced into the chamber 10 through a spray nozzle 56. The spray nozzle reintroduces the breathable liquid 12 to the chamber 10 in a manner that facilitates carbon dioxide purging. In alternative embodiments the breathable liquid 12 can be reintroduced into the chamber 10 without the aid of a spray nozzle 56.

In a more preferred embodiment, the chamber 10 also can include hold up screens configured to contact with the reintroduced, expired breathable liquid 12 as it enters the chamber 10. The screens break up the flow of liquid 12 and facilitate the liquid-oxygen contact, contributing to oxygenation and carbon dioxide purging. In one embodiment the screens attach to a rod in the chamber. One skilled in the art can easily select screens or like devices that can function in the selected chamber 10.

In another embodiment a heating device warms the breathable liquid to a desired temperature. For example, the temperature can be body temperature. In one embodiment, the heating device may be located “in-line” on one of the conduits 44, 54. In a preferred embodiment, the heating device may be part of or included in the chamber 10. For example, the same rod used to attach to the hold-up screens may also contain the heating device. The device may be an off the shelf commercial or custom made electrical version, and can be controlled manually or by a temperature controller. In automated or computer controlled embodiments, the temperature can be controlled by a control system as depicted below in FIG. 3.

A more preferred embodiment includes a safety system. The safety system releases pressure in the event of an emergency, such as a valve malfunction for example. The chamber 10 can include a rupture device, such as a disk or valve that opens at a specific pressure. As depicted in FIG. 2, the system can comprise a safety conduit 76 with a safety valve 80. The valve 80 can be closed during inspiration and expiration. The valve 80 can be controlled by circuitry such that it opens if the system “crashes.” The valve 80 can open if the pressure meets or exceeds a high threshold or meets or falls below a low threshold. The circuitry can independently operate the valve 80 even in the event of other circuit or computer malfunctions. One skilled in the art will recognize that other types of valves and that other safety system configurations can be used in the present invention.

In embodiments of the present invention the liquid contacting components can be “disposable” or single use. The liquid contacting components can include, for example, the chamber 10, the conduits 44, 54, 72, 76 as well as any filters, traps, condensers, and like components that contact with the breathable liquid 12. This can eliminate the need for cleaning of the components. Thus, greater sterility can be ensured. Further, the components of the apparatus can be disposed of after use by a single patient.

Additional breathable liquid can be added to the apparatus through a liquid introduction port according to methods well known to those of skill in the art. The breathable liquid can be added at any appropriate location on the apparatus such that sterility and pressure within the system are not compromised. For example, in one embodiment additional breathable liquid is added through a stopcock. In another embodiment additional breathable liquid is added via a three way valve. The additional breathable liquid can be added after total liquid ventilation is initiated.

In one preferred embodiment, the present invention can be automated through the use of a control system 82, as illustrated in FIG. 3. The control system 82 may advantageously be a computerized control system, implemented, for example, through a dedicated or a general purpose computer or microcontroller. In one nonlimiting embodiment, the control system monitors and controls the flow controllers 36, 40 in conjunction with the valves 50, 66, to facilitate tidal liquid breathing by the patient. For example, the control system 82 may be programmed to close the valve 66, open the valve 50, and increase flow through the first flow controller 36 (and/or decrease flow through the second flow controller 40) to initiate inspiratory flow of breathable liquid into the patient through the endotracheal tube 52. Then, when the control system 82 determines (e.g., through flow measurement, timing, or measurement of the volume of liquid in the chamber 10) that sufficient liquid has entered the patient during inspiration, the process can be reversed. The control system can close the first valve 50, open the second valve 64, and depressurize the chamber 10 by reducing flow through the first flow controller 36 and/or increasing flow through the second flow controller 40, thus initiating expiratory flow of fluid out of the endotracheal tube 52 and out of the patient. This process is repeated with each breathing cycle. Note that if the valves 50, 66 are check valves, then the opening and closing of the valves is automatic, without intervention of the control system 82. Control systems capable of use with embodiments of the present invention can be easily selected by those skilled in the art. The systems can control and interact with numerous components and parts beyond those depicted in FIG. 3.

The control system 82 also can interface with a pressure transducer 84. The pressure transducer 84 can be configured to measure the differential pressure between the bottom and the top of the chamber 10. In such a case, this differential pressure would correlate with the hydrostatic head in the chamber 10 and would be a method for measuring the amount of fluid in the chamber 10. Further, the rate of change of the differential pressure would correlate with the flow rate of liquid into and out of the lungs of a patient 16. If desired, another pressure transducer (not shown) may also measure the absolute pressure in the chamber 10 and convey that information to the control system 82. In one embodiment, a 0-5 PSI differential pressure transducer 84 can be used.

Another embodiment of the present invention relates to a method for providing total liquid ventilation to a patient. The method can include providing a liquid ventilation apparatus having a chamber containing a volume of a breathable liquid; delivering the breathable liquid from the liquid ventilation apparatus to a patient by pressurizing the chamber; and then moving the breathable liquid from the patient to the liquid ventilation apparatus by depressurizing the chamber.

The liquid ventilation apparatus can be, for example, the apparatus described above in relation to FIGS. 1-3. One skilled in the art can easily select other alternative liquid ventilation apparatuses based upon skill known in the art. As discussed above the breathable liquid can include any gas dissolving liquid or like material. In preferred embodiments the breathable liquid can include a fluorocarbon. Specific embodiments of suitable gas carrying liquids are well known to those of skill in the art.

The volume of the breathable liquid can depend upon the particular patient. The required volume for the system also can depend upon the volume required to prime the system. Where the distance from the liquid ventilation apparatus to the patient is short, then less volume is required. Conversely, where the distance is greater, then a larger volume of breathable liquid can be required. Thus, in preferred embodiments the minimum required volume can be the functional residual capacity of the patient plus the priming volume of the system, and may also include a safety factor of 10%, 20%, 30%, 40%, 50% or more of the functional residual capacity. Specifically, in some embodiments the volume is from 50 to 3000 mL. In other preferred embodiments the volume of breathable liquid can be approximately equal to at least the functional residual capacity of the patient, a priming volume, and a tidal volume. In another embodiment the volume of breathable liquid can be approximately equal to the functional residual capacity of the patient.

As mentioned above, the breathable liquid can be delivered from the liquid ventilation apparatus to a patient by pressurizing the chamber; and then moving the breathable liquid from the patient to the liquid ventilation apparatus by depressurizing the chamber or the apparatus. The pressurizing and depressurizing of the chamber or the apparatus can be accomplished by methods well known to those of skill in the art. In one embodiment, the pressurization and depressurization are accomplished utilizing the apparatus described above in relation to FIGS. 1-3.

Further, the delivery step can be accomplished through a first conduit and the moving step can be accomplished through a second conduit. As used herein, a conduit can be any type of pipe, channel, or other flexible or rigid device creating a passage through which the liquid may flow. In preferred embodiments the first or second conduit can include flexible tubing, such as urethane, viton, and the like. One of skill in the art easily can select suitable conduits based upon common skill in the art.

The flow of the breathable liquid can be selectively directed through the first or the second conduit by means of a valve. The valve can be a check valve or an externally actuated valve, and the like. The method can include valve configurations as described above, for example.

A further embodiment of the present invention relates to a method for performing full tidal liquid ventilation on a patient without the use of liquid pumps. The method can include pressurizing a breathable liquid-containing chamber by introducing oxygen containing breathing gas thereinto, thereby to move the breathable liquid into a patient; and depressurizing the chamber by removing gas therefrom, thereby to move breathable liquid from the patient into the chamber. For example, the method can be performed utilizing the apparatus described above, wherein breathable liquid is moved into and out of a patient without liquid pumps. The pressurization and depressurization can be accomplished as described above. Further, the pressurization and depressurization can be accomplished according to knowledge known by those of skill in the art.

Additionally, the breathable liquid can be moved from the chamber into the patient at least in part through a first conduit and the breathable liquid can be moved from the patient to the chamber at least in part through a second conduit. Suitable conduits for the present method are described above. The breathable liquid can be selectively directed through the first or the second conduit by a valve as described above and according to methods well known by those of skill in the art.

Although certain embodiments of the invention are featured in this disclosure, those embodiments are illustrative only, and the full scope of the present invention is defined by the following claims and permissible equivalents thereof:

What is claimed is:

1. A liquid ventilation apparatus for facilitating liquid breathing by a patient, comprising:
a chamber adapted to contain a breathable liquid and a gas;
a gas supply system connected to the chamber and adapted to supply an oxygen-carrying breathing gas into the chamber under pressure, thereby pressurizing the breathable liquid;
a gas removal system connected to the chamber and adapted to remove gas from the chamber;
a gas flow control system controlling the gas supply system and the gas removal system to alternately pressurize and depressurize the chamber;
a liquid supply line exiting the chamber and adapted to carry breathable liquid from the chamber to a patient when the chamber is pressurized; and
a liquid return line communicating with the chamber and adapted to carry breathable liquid from a patient to the chamber when the chamber is depressurized.

2. The apparatus of claim 1, further comprising at least a first valve associated with at least one of the liquid supply line and the liquid return line so that liquid flows only outward from the chamber through the liquid supply line or only inward toward the chamber through the liquid return line.

3. The apparatus of claim 2, wherein the first valve is a valve connected to both the liquid supply line and the liquid return line, and adapted to permit fluid communication between one, but not both, of said lines and the lungs of a patient at any one time.

4. The apparatus of claim 2, further comprising a second valve, wherein the first valve is a check valve controlling one-way flow through the liquid supply line and the second valve is a check valve controlling one-way flow through the liquid return line.

5. The apparatus of claim 2, further comprising a second valve, wherein the first valve is a pinch valve controlling one-way flow through the liquid supply line and the second valve is a pinch valve controlling one-way flow through the liquid return line.

6. The apparatus of claim 1, wherein pressurization of the chamber drives liquid flow out from the chamber through the liquid supply line and depressurization of the chamber draws liquid into the chamber through the liquid return line.

7. The apparatus of claim 1, wherein depressurization of the chamber reduces the pressure in the chamber to a level no greater than atmospheric pressure.

8. The apparatus of claim 1, wherein depressurization of the chamber reduces the pressure in the chamber to a pressure less than atmospheric pressure.

9. The apparatus of claim 1, wherein the gas flow control system comprises a flow controller adapted to control the flow of a gas into or out of the chamber.

10. The apparatus of claim 9, wherein the flow controller is a mass flow controller.

11. The apparatus of claim 1, further comprising a liquid conservation device.

12. The apparatus of claim 11, wherein the conservation device is associated with the gas removal system.

13. The apparatus of claim 1, further comprising a decontamination device for removing contaminants from the breathable liquid or gas.

14. The apparatus of claim 13, wherein the decontamination device is associated with the liquid supply line to decontaminate the breathable liquid.

15. The apparatus of claim 13, wherein the decontamination device is associated with the liquid return line to decontaminate the breathable liquid.

16. The apparatus of claim 1, wherein the chamber further comprises a spray nozzle by which the breathable liquid is reintroduced into the chamber such that gas exchange occurs.

17. The apparatus of claim 1, wherein the chamber comprises a gas diffuser connected to the gas supply system such that the oxygen-carrying breathing gas is diffused into the breathable liquid.

18. The apparatus of claim 1, wherein the chamber comprises a hold-up device to facilitate gas exchange.

19. The apparatus of claim 1, wherein the chamber is a column gas exchanger.

20. The apparatus of claim 1, comprising a volume of the breathable liquid.

21. The apparatus of claim 1, wherein the breathable liquid is a perfluorochemical (PFC).

22. The apparatus of claim 21, wherein the PFC is selected from the group consisting of FC-75, FC-77, RM-101, Hostinert 130, APF-145, APF-140, APF-125, perfluorodecalin, perfluorooctyl bromide, perfluorobutyltetrahydrofuran, perfluoropropyl-tetrahydropyran, dimethyladamantane, trimethyl-bicyclo-nonane, and mixtures thereof.

23. The apparatus of claim 1, wherein the oxygen containing breathing gas includes oxygen as a first gas and a second gas selected from the group consisting of nitrogen, helium, argon, or nitric oxide.

24. The apparatus of claim 1, wherein the gas is a bioactive agent or respiratory promoter.

25. The apparatus of claim 1, wherein the gas removal system comprises a vacuum source.

26. The apparatus of claim 1, further comprising an endotracheal tube for delivering the breathable liquid to the patient and for conveying the breathable liquid from the patient.

27. The apparatus of claim 1, further comprising an automated control system configured to control the activity of a valve or a flow control system.

28. The apparatus of claim 27, wherein the control system comprises a computer or other programmable controller.

29. The apparatus of claim 1, wherein the chamber is a reservoir and a gas exchanger.

30. A method for providing total liquid ventilation to a patient, comprising:
providing a liquid ventilation apparatus having a chamber containing a volume of a breathable liquid;
delivering the breathable liquid from the liquid ventilation apparatus to a patient by pressurizing the chamber; and then
moving the breathable liquid from the patient to the liquid ventilation apparatus by depressurizing the chamber.

31. The method of claim 30, wherein the delivery step is accomplished through a first conduit and the moving step is accomplished through a second conduit.

32. The method of 31, wherein the flow of the breathable liquid is selectively directed through the first or the second conduit by means of a valve.

33. The method of claim 32, wherein the valve is a check valve or an externally actuated valve.

34. A method for performing full tidal liquid ventilation on a patient without the use of liquid pumps, comprising:
pressurizing a breathable liquid-containing chamber by introducing oxygen containing breathing gas thereinto, thereby to move the breathable liquid into a patient; and
depressurizing the chamber by removing gas therefrom, thereby to move breathable liquid from the patient into the chamber.

35. The method of claim 34, wherein the breathable liquid is moved from the chamber into the patient at least in part through a first conduit and the breathable liquid is moved from the patient to the chamber at least in part through a second conduit.

36. The method of claim 35, wherein the breathable liquid is selectively directed through the first or the second conduit by a valve.
